(19) United States
(12) Patent Application Publication
(10) Pub. No.: US 2023/0053885 A1
(43) Pub. Date: Feb. 23, 2023

(54) MEDICAL DEVICE AND METHOD OF OPERATING A MEDICAL DEVICE

(71) Applicant: OLYMPUS WINTER & IBE GMBH, Hamburg (DE)

(72) Inventor: Stefan DIETRICH, Potsdam (DE)

(73) Assignee: OLYMPUS WINTER & IBE GMBH, Hamburg (DE)

(21) Appl. No.: 17/889,105

(22) Filed: Aug. 16, 2022

Related U.S. Application Data
(60) Provisional application No. 63/234,799, filed on Aug. 19, 2021.

(51) Int. Cl. A61M 60/873; H02J 7/02

(52) U.S. Cl. CPC A61M 60/873; H02J 7/02; H02J 2310/23

(57) ABSTRACT

A medical device and a method of operating a medical device, wherein the device includes a medical function assembly, a control assembly, a user interface, and a user information element, configured to display information about a type of the medical device, wherein the control assembly includes a memory element in which different operating parameters for the medical function assembly are storable and wherein the control assembly is configured to control the medical function assembly to perform different medical functions as a function of operating parameters stored in the memory element, wherein the control assembly is further configured to control the user information element to display information about the operating parameters stored in the memory element. The medical device wherein the user information element is configured to continue to reproduce the information after being controlled once by the control assembly, independent of continued control by the control assembly.

The invention relates to a medical device comprising a medical function assembly, a control assembly, a user interface, and a user information element, configured to display information about a type of the medical device, wherein the control assembly comprises a memory element in which different operating parameters for the medical function assembly are storable, and wherein the control assembly is configured to control the medical function assembly to perform different medical functions as a function of operating parameters stored in the memory element, wherein the control assembly is further configured to control the user information element to reproduce information about the operating parameters stored in the memory element.

The invention further relates to a method of operating a medical device.

Generic medical devices are used in modem medicine to provide complex medical functions. These can be diagnostic as well as interventional functions. Generic medical devices include, for example, electrosurgical radiofrequency and/or microwave generators, plasma generators, ultrasound generators, video endoscope controllers, ultrasound imaging devices, and tomography devices, although the foregoing list is not exhaustive.

The actual medical function of such medical devices can be controlled to a varying extent by operating parameters. For example, in the case of electrosurgical generators, the setting of operating parameters can be used to control whether a therapy signal provided is more suitable for cutting or coagulating tissue. In video endoscope controllers, operating parameters can be used to control various illumination modes and/or image enhancement methods. For tomography devices, operating parameters can be used to vary, for example, the orientation of imaging slices and the type of tissue types to be primarily imaged.

Because of the great importance of the stored operating parameters on the medical function of a generic medical device, it is necessary to reflect information about the operating parameters on a user information element so that a user of the medical device can check whether the correct operating parameters are stored for a planned examination or procedure. For this purpose, the corresponding information is usually shown on a display, for example a monitor, of the medical device. This requires the control assembly to continuously drive the display to reflect the information. This is inconvenient.

It is therefore an object of the invention to provide an improved medical device.

This object is achieved according to the invention by a medical device, comprising a medical function assembly, a control assembly, a user interface, and a user information element, configured to display information about a type of the medical device, wherein the control assembly comprises a memory element in which different operating parameters for the medical function assembly are storable, and wherein the control assembly is configured to control the medical function assembly to perform different medical functions as a function of operating parameters stored in the memory element, wherein the control assembly is further configured to control the user information element to reproduce information about the operating parameters stored in the memory element, which is further configured in that the user information element is configured to continue to reproduce the information after being controlled once by the control assembly, independent of continued control by the control assembly. The load on the control assembly can thus be reduced.

In an advantageous embodiment, the user information element may be configured to continue to reproduce the information after being controlled once by the control assembly, independent of an existing power supply to the medical device. In this way, it is possible to display the information about the stored operating parameters via the user information element even when the medical device is switched off and/or disconnected from a power supply. This can be useful, for example, if the stored operating parameters define a special medical function that deviates from the usual range of functions of the medical device. In this case, the corresponding embodiment allows the medical device modified in its functional scope to be distinguished from other, e.g. non-modified, medical devices of the same design even when switched off.

The user information element may comprise an electronic display. In this regard, the user information element may preferably comprise a power source independent of the power supply of the medical device.

For example, a primary battery and/or a secondary battery may be applicable to serve as an independent power source. For example, a primary battery may be designed to provide a continuous supply of power to the user information element over an intended use time or lifetime of the medical device. A secondary battery may be designed to be charged from the medical device power supply during a period of use of the medical device, and to be capable of permanently powering the user information element over an expected period of non-use.

In one possible embodiment of a medical device, the user information element may comprise a “memory in pixel” (MIP) display. In such displays, each pixel is associated with a separate semiconductor memory in which the activation state of the pixel is stored. MIP displays are characterized by extremely low power consumption, so that they can be operated for long periods of time using, for example, a primary or secondary battery.

In another possible embodiment, the user information element may comprise an e-paper display. Such displays comprise a plurality of microcapsules in which differently charged pigment particles in an emulsion can be aligned by applying a control voltage such that a visible side of each microcapsule assumes a predetermined gray or color value. The alignment of the pigments persists for a long time after the control voltage is removed, so that corresponding displays can continue to display an image once it has been set with very low power consumption or even completely without power consumption.

The operating parameters may include operating software. For the purposes of the invention, operating software means any form of machine-readable instructions that affect the operation of the medical function assembly. The operating software may directly and/or indirectly influence the performance of the medical function assembly. Indirect influence can be understood, for example, as influencing the operation of the control assembly, which in turn has an effect on the operation of the medical function assembly. In such a case, the information to be reproduced may comprise a version number or a variant number of the operating software.

According to another aspect of the invention, the object is achieved by a method of operating a medical device according to the above embodiments, comprising the steps of Providing the medical device, storing operating parameters in a memory element of the medical device, controlling a user information element of the medical device to reproduce information about the operating parameters stored in the memory element, and continuing the reproduction of the information by the user information element after the controlling has ended.

In this regard, the reproduction may continue independent of an existing power supply to the medical device. Furthermore, the operating parameters may comprise operating software.

With respect to the advantages and effects achievable thereby, explicit reference is made to what has been said above.

The invention will be explained in more detail below with reference to some exemplary figures and embodiments. In this context, the illustrated embodiment examples are intended solely for better understanding of the disclosure, without imposing any restrictions thereon.

The Figures show:

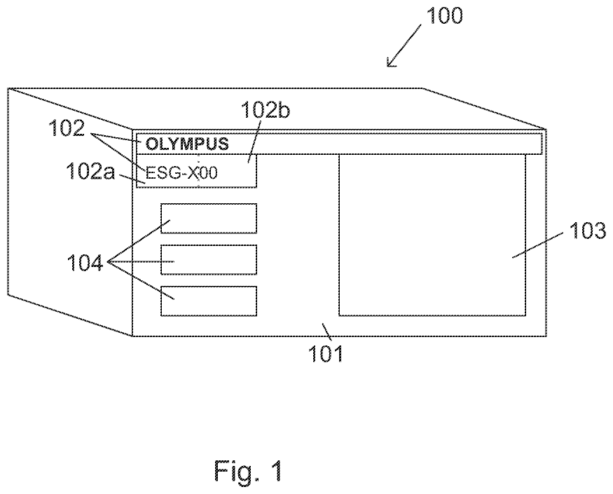

FIG. 1: A medical device,

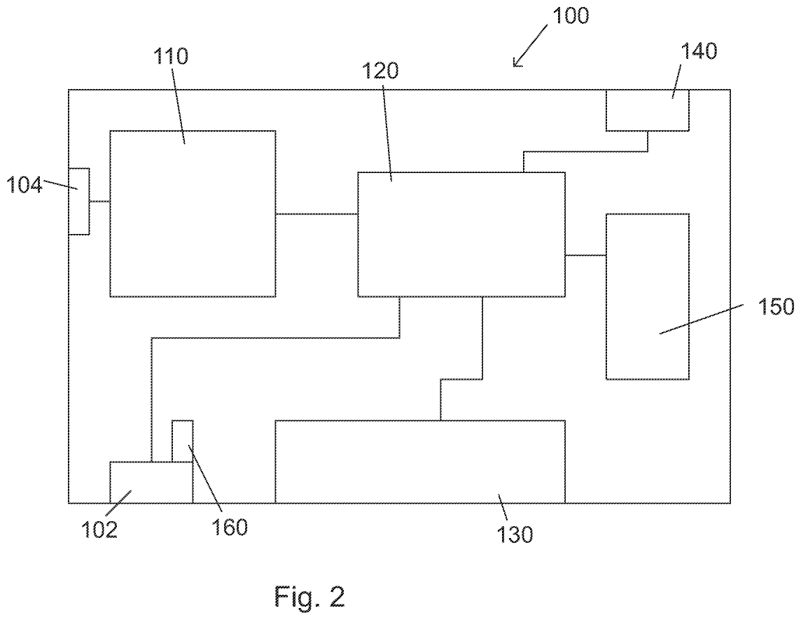

FIG. 2: A schematic representation of the structure of a medical device,

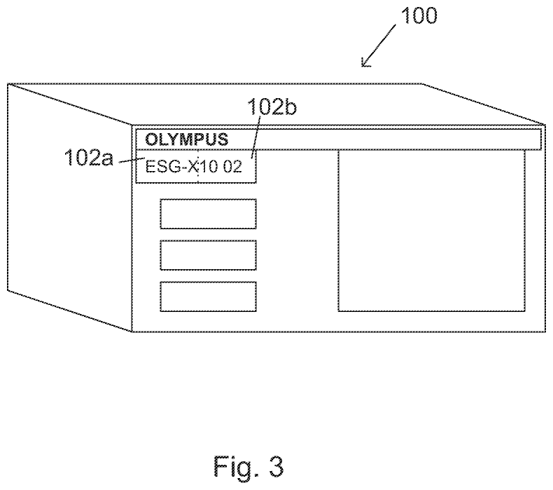

FIG. 3: The medical device of FIG. 1 with modified operating parameters,

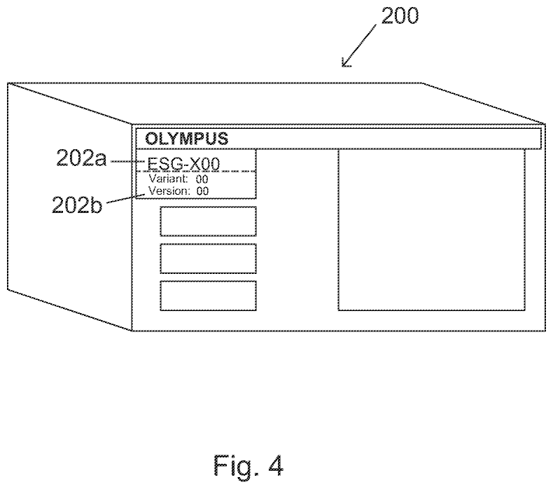

FIG. 4: Another medical device,

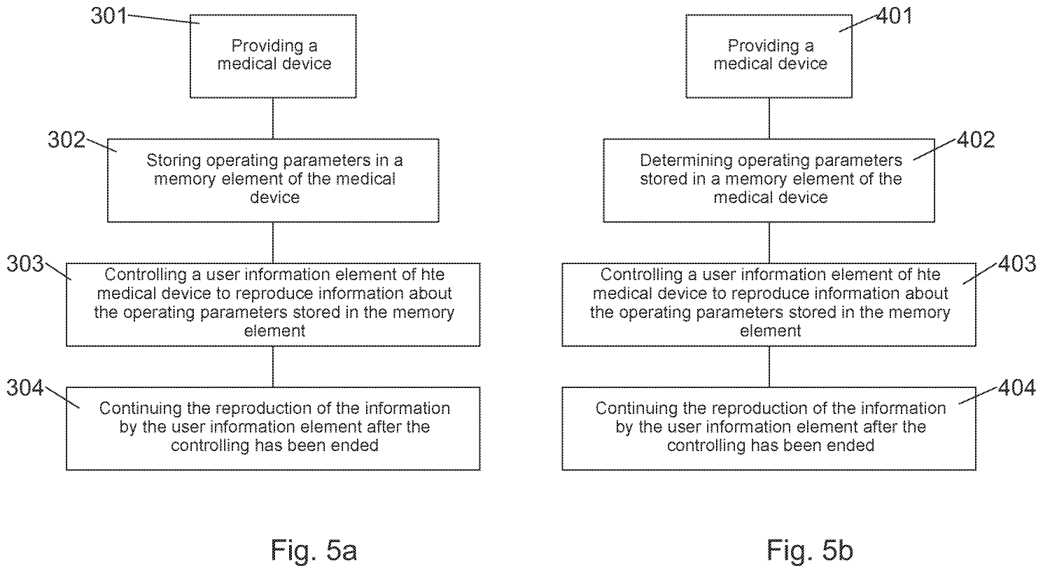

FIG. 5a, 5b: Method of operating a medical device.

FIG. 1 shows a medical device 100, in the example shown an electrosurgical generator.

On one front side 101 of the medical device a user information element 102 is arranged, which displays information about the type of the medical device. In the present example, the medical device is an “OLYMPUS ESG-X00” type generator. The user information element 120 includes a static area 102a and a dynamic area 102b, described in more detail below.

Furthermore, a screen 103, preferably a touch screen, and a plurality of ports 104 are provided on the front panel 101 of the medical device 100 to which electrosurgical instruments not shown can be connected to be operated with the medical device 100.

FIG. 2 schematically illustrates the internal structure of the medical device 100. A medical function assembly 110 provides the actual function, in the example shown, the provision of electrosurgical therapy signals. The medical function assembly 110 is connected to electrosurgical instrument ports 104. A control assembly 120 is provided to control the function assembly 110. A user interface 130 is provided to provide information to a user regarding the status of the medical function assembly 110 and the control assembly 120, and to receive user input. The user interface may include the display screen 103.

Further provided is a service interface 140 through which the control assembly 120 may send and receive data. The control assembly 120 is further connected to a memory element 150 in which operational data and operational parameters may be stored. The operational parameters of the medical device 100 stored in the memory element 150 include operating software that affects the operation of the control assembly 120 and/or the medical function assembly 110.

Changes may be made to the operating software via the service interface 140. For example, an updated version of the operating software may be stored in the memory element 150 to improve the operation of the medical device 100.

It is possible to modify the function of the medical device 100 to provide newly developed medical functions by storing a variant of the operating software. For example, this may provide compatibility of the medical device with a new electrosurgical instrument. In doing so, other medical functions may be omitted which cannot be provided by the new operating software.

In conventional medical devices, information about a stored variant or version of the operating software is output via the user interface when the medical device is switched on.

The medical device 100, on the other hand, enables the corresponding information to be displayed even when the device is switched off.

For this purpose, the user information element 102 comprises the dynamic area 102b. This area is designed as an electronic display and can be controlled by the control assembly to display information about the stored operating software. In this case, the display is designed in such a way that the respective information is permanently displayed after being controlled once by the control assembly 120, even if the medical device 100 is switched off and disconnected from the power supply.

The user information element 102 may be equipped with a power source 160 for this purpose, for example in the form of a primary or secondary battery. For example, if the dynamic area 102b of the user information element 102 is a memory-in-pixel (MIP) display, a single AA primary battery with a capacity of approximately 2000 mAh can easily power the display for a period of 10 years. MIP displays measuring approximately 3×3 cm, for example, are available with power consumption below 20 μW during still image playback. Alternatively, the power source 160 may be a rechargeable secondary battery, and may be recharged when the medical device 100 is in operation.

Instead of an MIP display, the dynamic area 102b may be implemented as an e-paper display. E-paper displays, unlike MIP displays, function with virtually no power supply during still image playback, and in this case, the power source 160 may not be required.

In the medical device 100 shown in FIG. 1, the static area 102a of the user information element 102 displays the information “OLYMPUS ESG-X”, while the dynamic area 102b of the user information element 102 displays the information “00”. Together, the areas 102a and 102b display the full type designation “OLYMPUS ESG-X00”. Therefore, when the medical device 100 is turned off, the user can see that the medical device 100 is equipped with standard operating software (variant 00).

In FIG. 3, the medical device 100 is shown after an alternative variant of the operating software (variant 10, version 02) has been stored in the memory element 150. Thus, the dynamic area 102b of the user information instrument 102 shows the information “10 02”.

Compounded, the user information element 102 now displays the changed type designation “OLYMPUS ESG-X10 02”, so that the user again recognizes without switching on the medical device 100 that the corresponding operating software has been loaded.

FIG. 4 shows a further medical device 200 whose structure, with the exception of the user information element, corresponds to the structure of the medical device 100 of FIGS. 1 to 3.

In the medical device 200, the user information element 200 is designed in such a way that the type designation “OLYMPUS ESG-X00” is shown completely on the static area 202a. Separate information on the variant and version of the installed operating software is reproduced on the dynamic area 202b in this case. Again, when the medical device 200 is turned off, a user can easily see which variant and version of the operating software are installed.

The embodiments described above show only two possibilities for an arrangement of the user information element on a medical device, and for the division into a static area and a dynamic area. In possible alternative embodiments, the user information element may be fully dynamic. In other possible embodiments, the user information element or its dynamic region may be implemented as part of a monitor of the medical device. Furthermore, a static area and a dynamic area of the user information element may be spatially separated from each other, and may be arranged, for example, on different side surfaces of the medical device.

FIGS. 5a and 5b illustrate methods for operating a medical device.

In the method illustrated in FIG. 5a, a medical device is first provided in step 301. The provision of the medical device comprises in particular the provision in an operational state, for example by establishing a power supply of the medical device and/or by switching on the medical device.

In a next step 302, operating parameters for the medical device are stored in a memory element of the medical device. This may be an initial storing of the operating parameters, for example at initialization or initial start-up of the medical device. It may also be a later storing of changed operating parameters of the medical device. The storing may take place, for example, via a service interface of the medical device. The operating parameters to be stored may be operating software of the medical device.

After storing the operating parameters, a user information element of the medical device is controlled in step 303 to reproduce information about the stored operating parameters. The control may be performed by a control assembly of the medical device.

Reproduction of the information by the user information element is then continued in step 304, even if the user information element is no longer controlled by the control assembly. Reproduction of the information may continue even if the medical device is turned off and/or power to the medical device is interrupted.

In the method illustrated in FIG. 5b, a medical device is again provided in step 401.

In step 402, operating parameters stored in a memory element of the medical device are then determined. In contrast to the method shown in FIG. 5a, no new or changed operating data is stored here. Rather, a control assembly determines, for example, which operating data are stored in the memory element.

In step 403, a user information element of the medical device is controlled to reproduce information about the stored operating parameters. The reproduction is then continued in step 404 even after the control of the user information element is terminated.

By the method shown in FIG. 5b, a reproduction of the information by the user information element may be refreshed when the medical device is put into operation. This takes into account the possibility that a quality of the reproduction by the user information element degrades after a longer period of time if no new control of the user information element takes place. Such degradation can occur in MIP displays due to random memory errors in the individual pixels. For e-paper displays, diffusion of the pigment particles in the microcapsules of the display may cause degradation.

1. A medical device, comprising a medical function assembly, a control assembly, a user interface, and a user information element configured to display information about a type of the medical device,
wherein the control assembly comprises a memory element, in which different operating parameters for the medical function assembly are storable, and wherein the control assembly is configured to control the medical function assembly to perform different medical functions as a function of operating parameters stored in the memory element,
wherein the control assembly is further configured to control the user information element to reproduce information about the operating parameters stored in the memory element,
wherein the user information element is configured to continue to reproduce the information after being controlled once by the control assembly, independent of continued control by the control assembly.

2. The medical device according to claim 1, wherein the user information element is configured to continue to reproduce the information after being controlled once by the control assembly, independent of an existing power supply of the medical device.

3. The medical device according to claim 2, wherein the user information element comprises an electronic display.

4. The medical device according to claim 3, wherein the user information element comprises a power source independent of the power supply of the medical device.

5. The medical device according to claim 4, wherein the power source comprises a primary battery.

6. The medical device according to claim 4, wherein the power source comprises a secondary battery.

7. The medical device according to claim 1, wherein the user information element comprises a “memory in pixel” display.

8. The medical device according to claim 1, wherein the user information element comprises an “e-paper” display.

9. The medical device according to claim 1, wherein the operating parameters comprise operating software.

10. A method of operating a medical device, comprising the steps of:
Providing the medical device,
Storing operating parameters in a memory element of the medical device,
Controlling a user information element of the medical device to reproduce information about the operating parameters stored in the memory element, and
Continuing the reproduction of the information by the user information element after the controlling has ended.

11. The method according to claim 10, wherein the reproduction is continued independent of an existing power supply of the medical device.

12. The method according to claim 10, wherein the operating parameters comprise operating software.
